SEQUENCE LISTING (1 sequence)

SEQ ID NO: 1 - DNA, 57 bp, artificial sequence
  mat_peptide: an added in coding sequence between the last coding codon of the chimeric Ad5/Ad3 fiber gene and its stop codon
ccatcagcctccgcatctgcttccgcccctagaggatcccatcaccatcaccatcac

(19) United States
(12) Patent Application Publication
(10) Pub. No.: US 2002/0151069 A1
(43) Pub. Date: Oct. 17, 2002

(54) Mosaic adenoviral vectors

(76) Inventor: Nikolay Korokhov, Birmingham, AL (US)

Correspondence Address: Benjamin Aaron Adler, ADLER & ASSOCIATES, 8011 Candle Lane, Houston, TX, 77071, US

(21) Appl. No.: 10/124,796

(22) Filed: Apr. 17, 2002

Related U.S. Application Data
(60) Provisional application No. 60/284,331, filed on Apr. 17, 2001.

(51) Int. Cl. C12N 15/861; C12N 7/00; C12N 5/06; C12P 21/02

(52) U.S. Cl. 435/456; 435/235.1; 435/69.1; 435/320.1; 435/325

(57) ABSTRACT

The present invention provides adenoviral vectors (Ad) that incorporate multiple distinct capsid modifications in a single virus particle, resulting in Ad that have improved gene delivery capacities and/or vector function.

### CROSS-REFERENCE TO RELATED APPLICATION

This non-provisional patent application claims benefit of provisional patent application U.S. Serial No. 60/284,331, filed Apr. 17, 2001, now abandoned.

### BACKGROUND OF THE INVENTION

1. Field of the Invention

The present invention relates generally to the field of adenovirus vectors. More specifically, the present invention relates to adenoviral vectors that incorporate multiple distinct capsid modifications.

2. Description of the Related Art

The human adenoviruses of serotype 5 (Ad5) is the most commonly used vector for gene therapy applications. Its utility as a gene delivery vehicle is largely based on its ability to infect a wide range of cell types with a remarkable efficiency (1).

There are, however, some limiting features for the use of Ad5 vectors for gene therapy. First of all, the widespread distribution of the adenoviral primary receptor—the coxsackievirus and adenovirus receptor (CAR)—precludes specific gene delivery to target cells. Furthermore, often the very cell types that are to be targeted, such as tumor cells, lack CAR and are therefore not permissive for infection by non-targeted adenovirus (1, 2).

In order to address these limitations, there have been various attempts to modify viral tropism with the ultimate intention to achieve both more efficient and more specific infection to target tissues and cells (1). For example, strategies have been endeavored to modify the native trophism of adenovirus to allow CAR-independent infection. Such CAR-independence of target cell binding/entry predicates increased gene transfer efficiency. A variety of strategies have been proposed to achieve adenovirus trophism modification including the employment of heterologous molecules, termed “re-targeting complexes”, which cross-link the adenovirus to non-CAR receptors. In addition, genetic modifications of the adenovirus capsid have been shown to accomplish the same end. In both instances, initial anchoring of the adenovirus to a non-native receptor is not inconsistent with target cell binding/entry followed by effective gene delivery. Indeed, it has been shown that it is possible to route adenovirus via a wide variety of heterologous cellular pathways. In many of these instances, retargeted entry can allow dramatic enhancements of adenovirus gene transfer efficiency via the circumvention of target cell CAR deficiency.

For practical gene therapy applications, the genetic capsid modification approach to trophism modification offers several advantages. This approach allows the achievement of CAR-independent gene delivery via diverse mechanisms. Heterologous targeting peptides have been incorporated into the HI loop (3-5) and COOH terminus (6-9) of the fiber protein, the penton base, hexon, and the minor capsid proteins, pIIIa and pIX. In addition, it has been shown that selected adenovirus serotypes achieve entry via distinct receptors different from that used by serotype 5, the serotype of the widely used adenoviral vector. On this basis, serotype chimerism for the fiber knob or for the entire fiber has allowed routing of the virus into non-CAR pathways.

It is noteworthy that in vivo gene delivery may be affected by factors over-and-above target cell adenovirus receptor levels. Specifically, the ability of adenovirus particles to transit in the context of anatomic barriers can affect in vivo efficacy. Thus, modulating the length of the fiber shaft, a maneuver which effects particle size, and thus, its distribution physiology, has resulted in altered in vivo gene delivery profiles. Moreover, genetic capsid alterations to modify particle charge may affect in vivo gene delivery dynamics. Therefore, these distinct strategies—incorporation of heterologous targeting peptides, capsid protein chimerism, fiber shaft modulation, and capsid charge modulation—can allow trophism alteration of adenovirus with the achievement of improved gene delivery dynamics.

Although the modifications in the adenoviral capsid mentioned above can achieve corresponding alteration in trophism, it has not been shown such alterations may be achieved in combination, resulting in additive or synergistic improvements in gene delivery and/or vector function.

Thus, the prior art is deficient in adenoviral vectors that incorporate multiple distinct capsid modifications to achieve altered trophism and enhanced gene delivery capacities. The present invention fulfills this long-standing need and desire in the art.

### SUMMARY OF THE INVENTION

The present invention provides adenoviral vectors (Ad) that incorporate multiple distinct capsid modifications such as incorporation of heterologous targeting ligand, capsid protein chimerism, fiber shaft modulation and capsid charge modulation. The resulting Ad would have improved gene delivery capacities and/or vector function.

In one embodiment of the present invention, there is provided an adenoviral vector comprising a heterologous targeting ligand incorporated into more than one capsid protein selected from the group consisting of hexon, fiber protein, p3 protein, p9 protein and penton.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows the design and analysis of a modified Ad3 vectors. FIG. 1A is a map of Ad5.F5/3.Ct.His, showing the localization of a short peptide linker (P(SA)₄P) and a six-His containing peptide (RGDSH₆) on the carboxy-terminus of the Ad3 fiber knob. The GFP and LUC expression cassettes are also indicated. Vector Ad5.F5/3 is essentially the same, except that it lacks the sequence encoding the peptide addition. FIG. 1B shows the confirmation of fiber region of the viral genomes by PCR. PCR 1 resulted in expected amplification products of 756 bp (lane 1) and 813 bp (lane 2) for Ad5.F5/3 and Ad5.F5/3.Ct.His respectively. PCR 2 resulted in amplification products of 138 bp (lane 1) and 195 bp (lane 2) for Ad5.F5/3. and Ad5.F5/3.Ct.His respectively. Lane M: 1 kb ladder.

FIG. 2 shows Western blot analysis of the fiber proteins of denatured Ad5.F5/3 (lane 1) and Ad5.F5/3.Ct.His (lane 2). FIG. 2A shows verification of fiber lengths by detection with anti-Ad5 fiber tail mAb 4D2. The fibers of Ad5.F5/3.Ct.His are of expected length, i.e. slightly larger than the fibers of Ad5.F5/3. FIG. 2B shows verification of presence of the His tag on the fibers of Ad5.F5/3.Ct.His by detection with anti-five-His monoclonal antibody. Size markers are indicated in kDa.

FIG. 3 shows binding of anti-five-His monoclonal antibody to Ad5.F5/3.Ct.His, but not to Ad5.F5/3, thus demonstrating the accessibility of the His tag on viral particles of Ad5.F5/3.Ct.His. A dilution range of virus immobilized in the wells of an ELISA plate was incubated with anti-five-His mAb and subsequently with an alkaline phosphatase conjugate for detection. Results are the mean of triplicate experiments.

FIG. 4 shows dose dependent inhibition by imidazole of Ad5.F5/3.Ct.His-mediated, but not Ad5.F5/3-mediated, gene transfer to U118MG-HissFv.rec cells, demonstrating that Ad5.F5/3.Ct.His is capable of mediating gene transfer via specific interaction between the His tag and the artificial His-tag receptor. Prior to infection for 30 min with the respective virus (MOI=100 virus particles per cell), the U118MG-HissFv.rec cells expressing AR were incubated for 10 min at room temperature with 0, 2.5 or 25 mM imidazole in PBS. Luciferase activities detected in the lysates of infected cells 24 hours post-infection are given as percentages of the activity in the absence of imidazole. Results are the mean of quadruplicate experiments.

### DETAILED DESCRIPTION OF THE INVENTION

The target cell binding of adenoviral vector (Ad) can be changed in a number of different ways so as to provide a means to circumvent the relative deficiencies of the serotype 5 receptor CAR. Altered target cell binding may be achieved via incorporation of heterologous targeting ligands within various distinct capsid proteins, or achieved via chimerisms of the adenoviral capsid by incorporating non-serotype 5 capsid components into Ad5-based vectors. Moreover, adenoviral capsid alterations may affect gene transfer efficiency by means other than altered target cell receptor recognition. Altered particle size or charge can affect interaction with anatomic barriers, and thus alter in vivo delivery efficiency. It is thus clear that genetic capsid modifications involving various distinct alterations of adenoviral biology such as incorporation of heterologous targeting peptides, capsid protein chimerism, fiber shaft modulation, and capsid charge modulation may be used to enhance in vivo adenovirus gene transfer efficiency.

Whereas these directed modifications in the adenoviral capsid can achieve corresponding alteration in trophism, it has not been appreciated that such alterations may be achieved in combination, resulting in additive or synergistic improvements in gene delivery and/or vector function. The present invention thus presents a novel paradigm of adenoviral trophism modification based on simultaneous incorporation of multiple distinct capsid modifications. This “complex mosaic” strategy would exploit the benefits of the various component modification strategies in the context of a single vector particle, which thus embodies the advantages of the contributing alterations. In addition to additive effects, various possibilities for functional synergy may also accrue in this general approach.

The present invention thus demonstrates that it is feasible to incorporate multiple distinct capsid modification within a single vector, termed “complex mosaic” particle, which provides a basis of improved gene delivery capacities/vector function compared to an adenovirus which is altered on a single capsid site. These mosaic designs may include, but are not limited to, the following modifications:

1) serotype chimerism and incorporation of heterologous ligand;

2) serotype chimerism of more that one capsid protein;

3) incorporation of heterologous ligands at more that one capsid focus;

4) altered fiber shaft length in combination with any, or all, of the above;

5) alterations specifically designed to modify the charge of adenovirus, in combination with any or all, of the above.

As used herein, the terms “serotype chimerism” refers to a virus with capsid proteins derived from multiple distinct serotypes.

As used herein, the term “capsid protein chimerism” refers to a capsid protein containing components derived from multiple distinct serotypes.

As used herein, the terms “knob serotype chimerism” refers to a virus with fiber knobs derived from multiple distinct serotypes.

As used herein, the terms “heterologous targeting ligand” refers to a binding moiety that can attach the virus to non-native receptor.

The present invention provides an adenoviral vector comprising a heterologous targeting ligand incorporated into more than one capsid protein, or more than one heterologous targeting ligand incorporated into more than one capsid protein. The capsid protein can be a hexon, fiber protien, p3 protein, p9 protein or penton. In general, the targeting ligands are physiologic peptide ligands, phase displayed peptide ligands, single chain antibodies (scFv) or components of single chain antibodies such as V_{H} and CDR3 regions of the single chain antibody.

The present invention also provides an adenoviral vector comprising more than one modified capsid protein such as hexon, fiber protein, p3 protein, p9 protein or penton, wherein said capsid proteins are modified by replacement with capsid proteins from another serotype.

The present invention also provides an adenoviral vector comprising a heterologous targeting ligand incorporated into one or more capsid protein such as hexon, fiber protein, p3 protein, p9 protein or penton, wherein the length of the fiber shaft of the adenoviral vector is altered.

The present invention also provides an adenoviral vector comprising a heterologous targeting ligand and more than one modified capsid protein such as hexon, fiber protein, p3 protein, p9 protein or penton, wherein the capsid proteins are modified by replacement with capsid proteins from another serotype.

The present invention also provides an adenoviral vector comprising more than one modified capsid protein such as hexon, fiber protein, p3 protein, p9 protein or penton, wherein said capsid proteins are modified by replacement with capsid proteins from another serotype, and wherein the length of the fiber shaft of the adenoviral vector is altered.

The present invention also provides an adenoviral vector which is charge-altered as a result of capsid modification, wherein said adenoviral vector also contains a modification such as incorporation of a heterologous targeting ligand, an altered fiber shaft length, or a capsid protein modified by replacement with capsid protein from another serotype.

The present invention also provides an adenoviral vector comprising more than one of the modifications selected from the group consisting of: a) a heterologous targeting ligand; b) a fiber shaft with altered length; c) capsid modification that results in charge alteration of said adenoviral vector; and d) capsid protein modified by replacement with capsid protein from another serotype.

The following examples are given for the purpose of illustrating various embodiments of the invention and are not meant to limit the present invention in any fashion.

### EXAMPLE 1

### Knob Serotype Chimerism Can Alter Ad Trophism and Enhance Ad Infectivity

A variety of target cells are adenovirus resistant based on a deficiency of the primary receptor for serotype 5 adenovirus. This is especially evident in the context of tumor cells, whereby CAR deficiency limits adenovirus vector efficiency, and thus the overall therapeutic potential of cancer gene therapy.

Adenovirus 3 and adenovirus 37 have been reported to recognize non-CAR receptors. On this basis, Ad5 vectors with knob chimerism for type 3 and 37 were derived. These vectors have been shown to be capable of enhanced infectivity of tumor cell compared to the type 5 adenovirus. These data thus establish the basis of knob chimerism as a means to alter adenoviral trophism, circumvent target cell CAR deficiency, and enhance adenoviral infectivity.

### EXAMPLE 2

### Adenoviral Vector Containing an Addition of a Heterologous Ligand to the Adenovirus Serotype 3 Fiber Knob

There is an increased interest in usage—for gene therapy purposes—of the adenovirus serotype 3 (Ad3) fiber knob, the structure of which only recently has been presented (10). Adenovirus 3 is a non-CAR binding serotype of adenovirus with a tropism distinct from Ad5 (11-14). In general, adenoviral cell tropism is regarded to be largely dependent on the initial binding event of the adenoviral fiber knob domain to a cognate cellular receptor. In case of Ad5 this receptor is CAR; however, for Ad3 an as yet unknown cellular receptor exists (11, 13-15).

Several studies have demonstrated that adenovirus tropism can be modified by replacing the fiber, or the fiber knob region, by that of another adenovirus serotype (12, 16-18). In this regard, it was shown that Ad5 based vectors carrying the Ad3 fiber knob, exhibit an Ad3 type tropism (12, 19). It has become apparent that some clinically relevant tissues exhibit differential expression of Ad3 and Ad5 receptors (19). Moreover, several target cell lines have been identified to which Ad3 receptor-mediated infection was more efficient than CAR-mediated infection (14, 19-20). On this basis, Ad3 tropism is also becoming of interest for gene therapy applications.

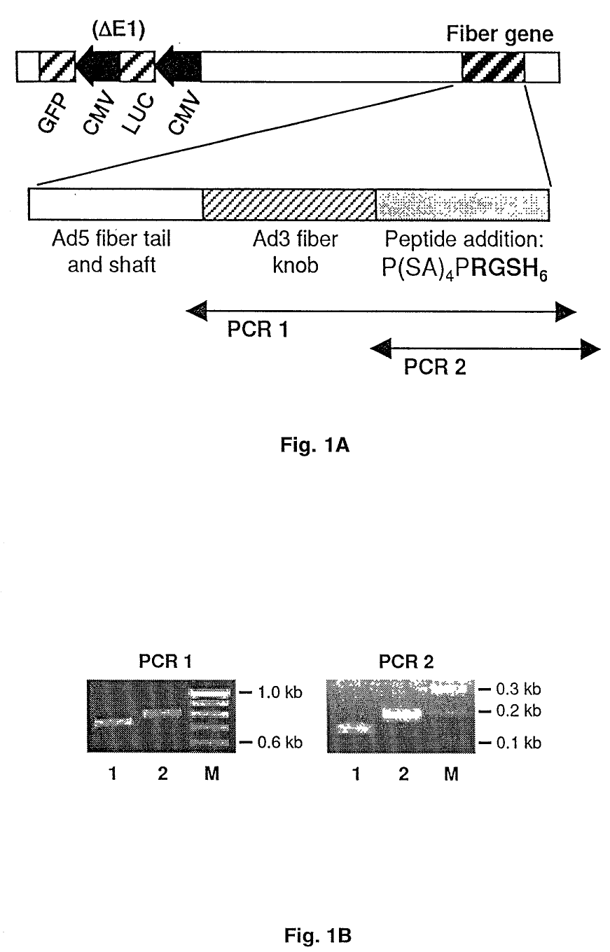

The present invention demonstrates that the carboxyl-terminus Ad3 fiber knob, like the Ad5 fiber knob, has suitable sites for incorporation of heterologous ligands. In the present example, two Ad5 based adenoviral vectors were modified by replacing the native fiber knob with an Ad3 fiber knob. These two vectors also contained within the E1 region an expression cassette consisting of a cytomegalovirus (CMV) promoter-driven green fluorescent protein (GFP) gene and a CMV promoter-driven firefly luciferase (LUC) gene (Ad5.F5/3 and Ad5.F5/3.Ct.His). Furthermore, in case of Ad5.F5/3.Ct.His, six His residues (preceded by a short spacer) had been genetically fused to the carboxy-terminus of the Ad3 fiber knob. Besides this ‘His-tag’ the two vectors were genetically the same (FIG. 1A).

These two modified vectors were constructed as follows: a plasmid containing the Ad5.F5/3 genome was generated by homologous DNA recombination between a PacI-KpnI fragment of pNEB.PK.F5/3 and a SwaI digested pVK50-8 based plasmid in E. coli BJ5183. pNEB.PK.F5/3 is a fiber shuttle vector containing a chimeric Ad5/Ad3 fiber gene (12), whereas the pVK50-8 based plasmid contained the aforementioned GFP and LUC expression cassette in the E1 region (21). A plasmid containing the Ad5.F5/3.Ct.His genome was generated in a similar manner, except that pNEB.PK.F5/3 had to be first modified so that a short peptide linker—Pro-(Ser-Ala)₄-Pro and a six-His containing peptide Arg-Gly-Ser-His₆ would be added to the carboxy-terminus of the chimeric Ad5/Ad3 fiber. To this end a PCR technique was employed that in resulted in the introduction of the coding sequence 5′-CCATCAGCCTCCGCATCTGCTTCCGCCCCTAGAG GATCCCATCACCATCACCATCAC-3′ (SEQ ID No. 1) between the last coding codon of the chimeric Ad5/Ad3 fiber gene and its stop codon.

Adenovirus DNA was released from the generated adenovirus genome plasmids by PacI digestion and used for transfection of 293 cells to rescue the virus as described previously (22). The viruses were rescued successfully, indicating that the heterologous addition to the Ad3 fiber knob was structurally compatible with correct folding and biological functions of the fiber molecule. The adenovirus vectors were propagated on 293 cells and purified by centrifugation in CsCl gradients by a standard protocol. Viral particle titers were determined spectrophotometrically by the method of Maizel et al. (23), using a conversion factor of 1.1×10¹² viral particles per absorbance unit at 260 nm.

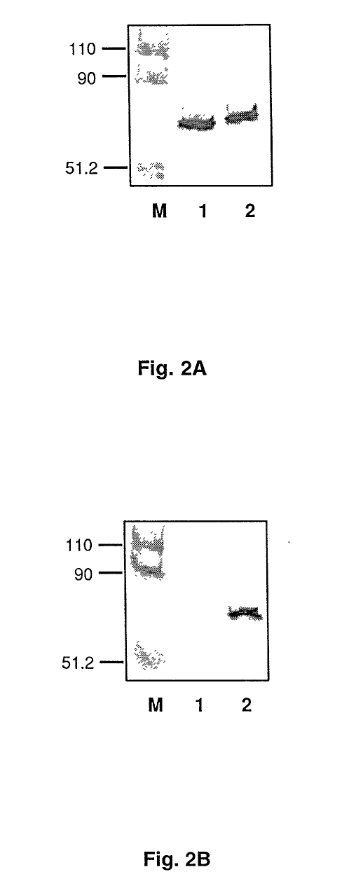

To verify the structural integrity of the fiber region of the viral genomes, DNA isolated from viral particles was analyzed by PCR. In both cases (Ad5.F5/3 and Ad5.F5/3.Ct.His) this resulted in the generation of amplification products of the expected lengths (FIG. 1B). Western blot (WB) analysis of denaturated viral particles demonstrated that the chimeric Ad5/Ad3 fibers had the predicted size (FIG. 2A). It was also verified that the carboxy-terminal His-tag was present on the fibers of Ad5.F5/3.Ct.His and absent on those of the control virus Ad5.F5/3 (FIG. 2B).

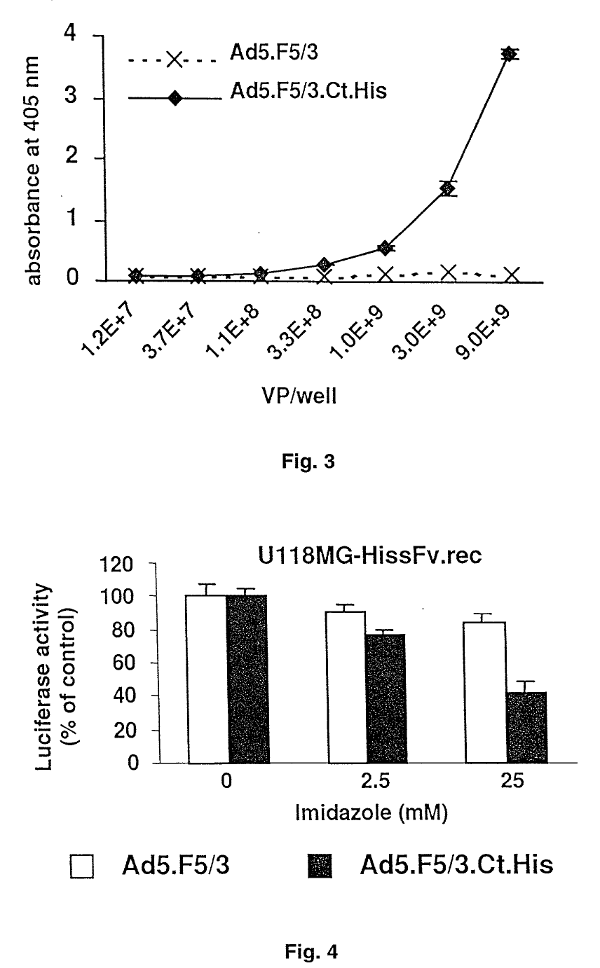

If the carboxy-terminus of the Ad3 fiber knob is to be used for re-targeting strategies, then it is of necessity that targeting moieties incorporated at this site are accessible for binding in the context of the intact virion. To investigate whether this was the case for the carboxy-terminal added His-tag, an enzyme-linked immunosorbent assay (ELISA) was performed. A range of three-fold dilutions of CsCl-purified virions (Ad5.F5/3 and Ad5.F5/3.Ct.His) immobilized in the wells of an ELISA plate were incubated with an anti-five-His mAb (Qiagen). Bound monoclonal antibody was detected by incubation with a goat anti-mouse IgG conjugated to alkaline phosphatase followed by development of the plate with p-nitrophenyl phosphate and reading at 405 nm. This analysis clearly showed efficient binding of anti-five-His antibody to immobilized particles of Ad5.F5/3.Ct.His, while binding to the control virus (Ad5.F5/3) was at the background level at every virus dilution (FIG. 3). These results demonstrate that the carboxy-terminal His-tags present on the Ad3 fiber knobs of intact virus particles were indeed accessible for binding and, therefore, potentially available for interaction with a cognate cell surface receptor.

Next it was determined whether the His tags on the Ad3 fiber knobs of Ad5.F5/3.Ct.His virions were capable of functioning as receptor-binding ligands and mediating gene transfer via a non-Ad3 receptor. This was addressed by Ad-mediated gene transfer assays (21) utilizing U118MG-HissFv.rec cells which exhibit surface expression of an artificial His-tag receptor (AR) with specificity for carboxy-terminal His-tags (24, 25). Specifically, a blocking experiment was conducted that capitalized on the fact that the artificial receptor has affinity (K_{D}=4×10⁻⁴ M) for imidazole (25). Results in FIG. 4 demonstrated that Ad5.F5/3.Ct.His gene transfer to U118MG-HissFv.rec cells was inhibited by imidazole in a dose-dependent manner, while this was not the case for Ad5.F5/3 gene transfer. This verifies that the modified virus, Ad5.F5/3.Ct.His, was indeed capable of infecting U118MG-HissFv.rec cells by means of a specific interaction between the carboxy-terminal His tag of the chimeric Ad5/Ad3 fiber protein and the artificial His-tag receptor.

In conclusion, the Ad3 fiber knob had not been previously explored for the presence of potential sites that can harbor heterologous targeting motifs. In the present example a heterologous ligand was added to the carboxy-terminus of the Ad3 fiber knob of an Ad vector. This genetic modification proved to have rendered the vector capable of mediating gene transfer via an alternate, non-Ad3 receptor. Thus, this work demonstrates that the carboxy-terminus of the Ad3 fiber knob is feasible as a locale for the introduction of novel tropism determinants.

### EXAMPLE 3

### Heterologous Targeting Peptides Can be Incorporated at Multiple Capsid Locales Within the Same Particles

It was shown previously that the targeting peptide RGD4C can be incorporated at the HI loop of the fiber knob. This modification allows CAR-independent gene delivery with efficiency enhancements. In addition, Vigne et al. has shown that this motif may be incorporated at the L loop of hexon with similar augmentations in gene transfer efficiency. On this basis, an adenovirus vector was constructed that incorporated this modification at both locales. The vector was constructed and rescued. The derivation of such a vector thus establishes the feasibility of deriving adenovirus vectors with “complex mosaic” configurations—that is incorporation of multiple distinct alteration within the same particle.

The following references are cited herein:

1. Russel, J. Gen. Virol. 81:2573-2604 (2000).

2. Pickles et al., J. Virol. 72:6014-6023 (1998).

3. Dmitriev et al., J. Virol. 72:9706-9713 (1998).

4. Krasnykh et al., J. Virol. 72:1844-1852 (1998).

5. Xia et al., J. Virol. 74:11359-11366 (2000).

6. Michael et al., Gene Ther. 2:660-668 (1995).

7. Wickham et al., J. Virol. 71:8221-8229 (1997).

8. Wickham et al., Nat. Biotechnol. 14:1570-1573 (1996).

9. Yoshida et al., Hum. Gene Ther. 9:2503-2515 (1998).

10. Durmort et al., Virology 285:302-312 (2001).

11. Defer et al., J. Virol. 64:3661-3673 (1990).

12. Krasnykh et al., J. Virol. 70:6839-6846 (1996).

13. Stevenson et al., J. Virol. 69:2850-2857 (1995).

14. Von Seggern et al., J. Virol. 74:354-362 (2000).

15. Roelvink et al., J. Virol. 72:7909-7915 (1998).

16. Gall et al., J. Virol. 70:2116-2123 (1996).

17. Shayakhmetov et al., J. Virol. 74:2567-2583 (2000).

18. Zabner et al., J. Virol. 73:8689-8695 (1999).

19. Stevenson et al., J. Virol. 71:4782-90 (1997).

20. Su et al., J. Vasc. Res. 38:471-478 (2001).

21. Seki et al., J. Virol. 76:1100-1108 (2002).

22. Chartier et al., J. Virol. 70:4805-4810 (1996).

23. Maizel et al., Virology 36:115-125 (1968).

24. Douglas et al., Nat. Biotechnol. 17:470-475 (1999).

25. Lindner et al., Biotechniques 22:140-149 (1997).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An adenoviral vector comprising a heterologous targeting ligand incorporated into more than one capsid protein selected from the group consisting of hexon, fiber protein, p3 protein, p9 protein and penton.

2. An adenoviral vector comprising more than one heterologous targeting ligand incorporated into more than one capsid protein selected from the group consisting of hexon, fiber protein, p3 protein, p9 protein and penton.

3. An adenoviral vector comprising more than one modified capsid protein selected from the group consisting of hexon, fiber protein, p3 protein, p9 protein and penton, wherein said capsid proteins are modified by replacement with capsid proteins from another serotype.

4. An adenoviral vector comprising a heterologous targeting ligand incorporated into one or more capsid protein selected from the group consisting of hexon, fiber protein, p3 protein, p9 protein and penton, wherein the length of the fiber shaft of said adenoviral vector is altered.

5. An adenoviral vector comprising a heterologous targeting ligand and more than one modified capsid protein selected from the group consisting of hexon, fiber protein, p3 protein, p9 protein and penton, wherein said capsid proteins are modified by replacement with capsid proteins from another serotype.

6. An adenoviral vector comprising more than one modified capsid protein selected from the group consisting of hexon, fiber protein, p3 protein, p9 protein and penton, wherein said capsid proteins are modified by replacement with capsid proteins from another serotype, and wherein the length of the fiber shaft of said adenoviral vector is altered.

7. An adenoviral vector which is charge-altered as a result of capsid modification, wherein said adenoviral vector also contains a modification selected from the group consisting of incorporating a heterologous targeting ligand, an altered fiber shaft length, and a capsid protein modified by replacement with capsid protein from another serotype.

8. The adenoviral vector of claim 7, wherein said capsid modification for charge alteration is selected from the group consisting of capsid addition, capsid deletion and capsid substitution.

9. The adenoviral vector of claim 7, wherein said capsid protein is selected from the group consisting of hexon, fiber protien, p3 protein, p9 protein and penton.

10. An adenoviral vector comprising at least one of the modifications selected from the group consisting of
a) addition of a heterologous targeting ligand;
b) a fiber shaft with altered length;
c) capsid modification that results in charge alteration of said adenoviral vector; and
d) capsid protein modified by replacement with capsid protein from another serotype.

11. The adenoviral vector of claim 10, wherein said capsid protein is selected from the group consisting of hexon, fiber protein, p3 protein, p9 protein and penton.

12. The adenoviral vector of claim 10, wherein said capsid modification for charge alteration is selected from the group consisting of capsid addition, capsid deletion and capsid substitution.
